(19) United States
(12) Patent Application Publication
(10) Pub. No.: US 2003/0204200 A1
(43) Pub. Date: Oct. 30, 2003

(54) Splash shield for use with debridement devices

(76) Inventor: Mark Rufener, Dover, OH (US)

Correspondence Address: BAKER & DANIELS, 111 E. WAYNE STREET, SUITE 800, FORT WAYNE, IN, 46802

(21) Appl. No.: 10/133,064

(22) Filed: Apr. 26, 2002

(51) Int. Cl. A61B 17/32

(52) U.S. Cl. 606/172

(57) ABSTRACT

A shield for use with wound debridement systems. The shield of the present invention includes a radially extending region terminating in a distal opening. The radially extending portion joins a neck having a proximal opening formed therein. A plurality of raised ribs extend into the proximal opening. The raised ribs are sized to create a press fit with a debridement tip inserted therein, with vent apertures being formed between adjacent ribs to prevent the formation of a vacuum and the resultant attachment of the shield to the tissue being debrided. The shield is manufactured from a substantially clear flexible plastic and with a generally flat surface, thereby facilitating assessment of the debridement procedure by a health care professional. Because a flexible plastic is utilized, the shield may be flexed and manipulated to fit the form of the patient and further allows for movement of a debridement tip operably positioned in the shield independent of movement of the shield.

### BACKGROUND OF THE INVENTION

1. Field of the Invention.

This invention relates generally to wound debridement devices, and, more particularly, to splash shields for use with such devices.

2. Description of the Related Art.

Debridement is the surgical excision of dead, devitalized, and contaminated tissue, and/or the removal of foreign matter from a wound. Hand-held wound debridement devices provide a viable option for wound debridement. Hand-held wound debridement devices, generally include an irrigation system to provide pressurized streams of fluid to dislodge unwanted tissue and foreign matter from a wound and a suction system to collect dislodged matter and irrigation fluid. However, the use of such systems can, disadvantageously, result in the splashing of irrigation fluid and body fluids from the patient onto, e.g, the health care professional operating the irrigation device.

Developments have been made to reduce the amount of splash generated from wound debridement devices utilizing irrigation fluid as the mechanism for debridement. Various shields or splash guards have been utilized in an effort to deflect excess irrigation and body fluids away from the health care professional operating the debridement device. Conventional splash deflection devices generally comprise structures formed about and extending outwardly from a debridement tip. These prior art devices suffer from numerous deficiencies. For example, the suction created by the debridement tip typically forms a sufficient vacuum to cause conventional splash deflection shields to adhere to the patient during the debridement procedure, resulting in ineffective irrigation of the wound and discomfort to the patient.

One conventional method of solving the problem of conventional splash shields adhering to patients provides one or more vent holes in the portion of the splash shield extending outwardly from the debridement tip. The vent holes of prior art devices are radially displaced from the debridement tip and seek to prevent the shield from adhering to the patient by preventing the formation of a vacuum between the shield and the patient. Problematically, the vent holes of conventional splash shields allow irrigation fluid and body fluids to seep and even spray out of the vent holes, thereby causing a marked decrease in the effectiveness of these prior art splash shields.

Conventional shield, or deflection devices, due to their materials of construction and their size and shape, often interfere with the user's ability to monitor the area being debrided. Additionally, conventional deflection devices are typically incorporated into a debridement tip and are not capable of being retrofit to alternative wound debridement devices.

What is needed in the art is a splash shield which is effective, yet which does not have a tendency to adhere itself to the patient undergoing debridement.

Also needed in the art is a splash deflection device capable of being retrofit to preexisting wound debridement devices.

Further needed in the art is a splash deflection device through which the affected area can be monitored during debridement.

### SUMMARY OF THE INVENTION

The present invention comprises a transparent shield having a substantially central aperture sized to accommodate a standard debridement tip, with at least one vent aperture being formed between the debridement tip and the shield. With a debridement tip traversing the central aperture of the shield of the present invention, the shield is operable to decrease splash generated during use of a debridement devise, while not being susceptible to vacuum adhering to the subject patient.

In one exemplary embodiment, the present invention comprises a shield having a conical region tapering into a neck having a substantially central aperture, or proximal opening formed therein. The conical region of the shield terminates in a distal opening. The proximal opening is sized to accommodate a standard debridement tip, such as, e.g., one of the numerous debridement tips utilized with the ZIMMER PULSAVAC® systems. In one exemplary embodiment, the proximal opening of the shield includes a plurality of interior, raised ribs forming contact points with the debridement tip, and thereby forming vent apertures between adjacent ribs to prevent the formation of a vacuum within the shield when the shield is applied to a patient

The exterior of the neck portion of the splash shield of the present invention may be textured, such that it provides a gripping surface for the operator. The shield is manufactured from a relatively flexible plastic, and therefore may be flexed and manipulated so that the distal end thereof fits the patient's form and thereby provides continuous contact with the patient to prevent seepage of irrigation fluid and body fluids between the patient and the shield. Additionally, the radially extending portion of the shield is relatively flat, and the plastic is substantially clear, thus allowing the operator to monitor the debridement procedure.

Advantageously, the splash deflection shield of the present invention is adapted to be retrofit to both newly manufactured and pre-existing wound debridement devices, and may be used with debridement tips having various spray patterns. Because the shield of the present invention may be used with such a wide variety of debridement devices, it is economical and convenient.

An object of the present invention is to provide a splash deflection shield which effectively deflects splash away from the operator and which does not adhere to the patient during use.

Another object of the present invention is to provide a splash deflection shield adapted to be retrofit to various pre-existing wound debridement devices.

Yet another object of the present invention is to provide a splash deflection shield for use with a debridement device which does not interfere with, or distort, the operator's view of the area being debrided.

### BRIEF DESCRIPTION OF THE DRAWINGS

The aforementioned and other features and objects of this invention, and the manner of attaining them, will become apparent and the invention itself will be better understood by reference to the following description of an embodiment of the invention taken in conjunction with the accompanying drawings, wherein:

FIG. 1 is a perspective view of the shield of the present invention;

FIG. 2 is a top elevational view thereof;

FIG. 3 is a radial elevational view thereof;

FIG. 4 is a cross-sectional view of the shield of the present invention, taken along line 4-4 of FIG. 2, illustrating placement of the shield about a debridement tip; and

FIG. 5 is an enlarged cross-sectional view of the neck region and raised ribs of the shield of the present invention.

### DETAILED DESCRIPTION OF THE INVENTION

The embodiment disclosed below is not intended to be exhaustive or to limit the invention to the precise form disclosed in the detailed description. Rather, the embodiments are chosen and described such that others skilled in the art might utilize their teachings.

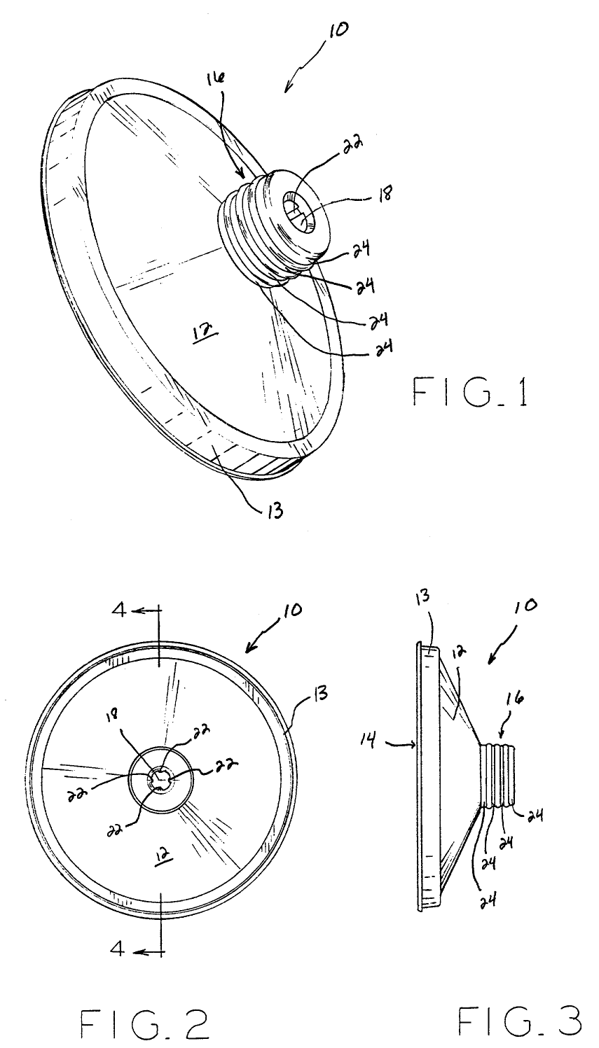

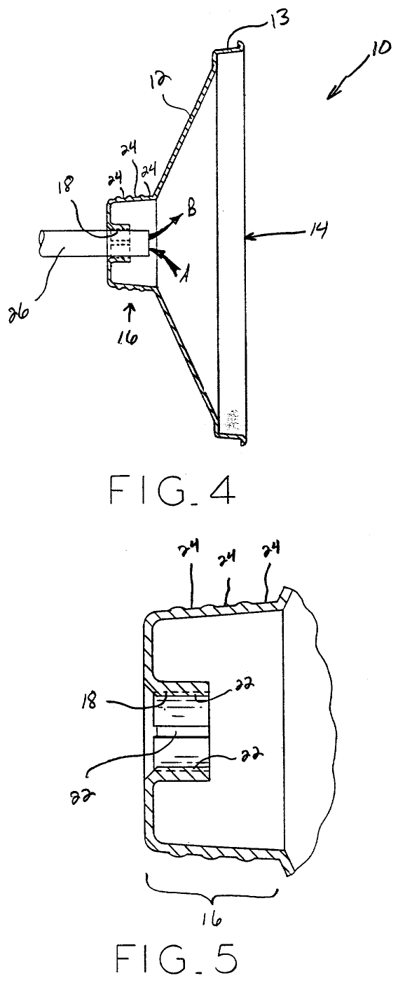

With reference to FIGS. 1-3, shield 10 includes radially extending portion 12 and neck region 16. Shield 10 further includes rim 13 disposed about the periphery of radially extending portion 12. Rim 13 terminates in distal opening 14, which is generally about 17.8 cm (7″) in diameter. Rim 13 may include a distal flange as illustrated in FIGS. 1-4. During use, rim 13 is placed in contact with or adjacent to the patient, and irrigation fluid flowing from debridement tip 26 (schematically depicted at “B” in FIG. 4) contacts the area to be debrided and thereafter is suctioned into debridement tip 26 as schematically depicted at “A” in FIG. 4 or is collected by shield 10. Fluid collected by shield 10 can be, e.g., allowed to drip from shield 10 into a collection device, or absorbed by an appropriately positioned sponge.

In one exemplary embodiment, radially extending portion 12 of shield 10 is conically shaped and tapers into neck 16. In one embodiment of the present invention, neck 16 is cylindically shaped. Neck 16 may also be conically shaped, e.g., tapering from a distal end thereof to a proximal end thereof. Neck 16 includes proximal opening 18, which, in one exemplary embodiment, is about 4.1 cm (1.6″) in diameter. Proximal opening 18 accommodates debridement tip 26 as illustrated in FIG. 4. As can be seen in FIGS. 1, 2, 4, and 5, a plurality of ribs 22 are disposed about the interior of proximal opening 18. Ribs 22 form the points of contact between shield 10 and debridement tip 26. Debridement tip 26 forms an interference or press fit with ribs 22. The spaces between adjacent ribs 22 form openings in the connection between the exterior of debridement tip 26 and the interior of neck 16, thereby providing a vent and preventing the formation of a vacuum and the attachment of shield 10 to a patient.

The vent apertures created by the cooperation of ribs 22 with debridement tip 26 provide several advantages over the vent apertures of prior art devices. As described above, prior art devices utilize vent apertures located a distance from the debridement tip in the radially extending portion of a shield formed about the debridement tip. In these prior art devices, irrigation fluid and body fluid contacting the shield near the periphery thereof could be directed over the vent hole in the radially extending portion of the shield by the suction created by the debridement tip. In this situation, the irrigation fluid and body fluid may disadvantageously exit the shield through the vent hole. Moreover, irrigation fluid streams provided by the debridement tip of prior art devices may deflect from the wound area and directly spray through the vent hole formed in the radially extending portion of the shield. Because the vent holes of the prior art devices are placed in the radially extending portions of the shield, they provide for a relatively large exit area for irrigation fluid and body fluid.

Contrary to the vent holes of the prior art devices, the vent holes formed between adjacent ribs 22 of the current invention are positioned adjacent the debridement tip, and, therefore, irrigation fluid and body fluid drawn toward or deflected toward debridement tip 26 will tend to be drawn into the suction portion of debridement tip 26 as opposed to exiting shield 10 through the apertures formed between adjacent ribs 22.

The external surface of neck region 16 provides a handle for user manipulation of shield 10. The external surface of neck 16 may be textured, such as with grooves 24, as shown in FIG. 1. Grooves 24 facilitate gripping of shield 10 by an operator.

Advantageously, shield 10 comprises a substantially transparent and flexible plastic material. In one exemplary embodiment, shield 10 comprises transparent polyvinylchloride (PVC) plastic having a Shore A durometer hardness of 90. Generally, shield 10 is formed of a material having a Shore A durometer hardness of about 80-95. The use of a flexible plastic is ideal because it allows shield 10 to be flexed and manipulated to, e.g., fit to the patient's form. Moreover, the flexibility of the materials of construction for shield 10 allow for pivoting movement of debridement tip 26 when positioned within proximal opening 18 thereof. The flexibility of shield 10 allows neck 16 to flex with debridement tip 26 while the perimeter of shield 10 remains operably positioned adjacent the subject patient. The surface of radially extending portion 12 is substantially flat and transparent, to permit the operator to monitor the debridement procedure.

FIG. 4 is a cross-sectional view of shield 10 with debridement tip 26 fit into proximal opening 18. Debridement tip 26 provides suction, as schematically depicted by Arrow A, and also provides irrigation fluid, as schematically depicted by Arrow B. Debridement tip 26 may be of virtually any type, so long as it is of a size and shape accommodated by proximal opening 18. It is contemplated that various shields 10 having proximal openings 18 of various sizes may be manufactured to accommodate debridement tips having outer diameters of varying sizes and geometries. Advantageously, shield 10 is capable of being utilized in conjunction with a wide variety of newly manufactured and preexisting debridement devices having various spray patterns.

Prior to debridement, tip 26 is inserted into proximal open end 18 of shield 10 and press fit with ribs 22 therein. As irrigation fluid is sprayed out of tip 22 onto the wound being debrided, irrigation fluid and bodily fluid can be drawn into the suction port in debridement tip 26 or collected by shield 10 as described herein-above.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which the invention pertains.

What is claimed is:

1. A splash shield for use with a debridement tip, comprising:
a shield body including a central aperture sized to accommodate the debridement tip, said shield body extending radially from said aperture; and
a rib extending into said central aperture.

2. The splash shield of claim 1, wherein said rib is sized to create a press fit with the debridement tip when the debridement tip traverses said central aperture.

3. The splash shield of claim 1, wherein said shield is substantially transparent.

4. The splash shield of claim 1, wherein said shield is manufactured from polyvinylchloride.

5. The splash shield of claim 11 wherein said shield has a Shore A durometer hardness of about 80-95

6. The splash shield of claim 5, wherein said shield has a Shore A durometer hardness of about 90.

7. The splash shield of claim 1, further comprising a rim extending substantially transversely from a perimeter of said shield.

8. A splash shield for use with a debridement tip, comprising:
a shield body having a substantially central aperture sized to accommodate the debridement tip, said shield body extending radially from said aperture, said shield formed of a material having a Shore A durometer hardness of about 80-95.

9. The splash shield of claim 8, wherein said shield has a Shore A durometer hardness of about 90.

10. The splash shield of claim 8, wherein said shield is substantially transparent.

11. The splash shield of claim 8, wherein said shield is formed from polyvinylchloride.

12. The splash shield of claim 8, further comprising a rim extending substantially transversely from a perimeter of said shield.

13. The splash shield of claim 8, further comprising a rib extending into said central aperture, said rib sized to create a press fit with the debridement tip when the debridement tip traverses said central aperture.

14. An apparatus for wound debridement, comprising:
a debridement tip, said debridement tip operable to direct an amount of irrigation fluid from a source of irrigation fluid onto an area; and
a splash shield including a central aperture sized to accommodate the debridement tip and a shield body extending radially from said aperture;
said central aperture of said shield having an inner diameter geometry, said debridement tip having an outer diameter geometry, whereby the inner diameter geometry of said central aperture is incongruent with the outer diameter geometry of said debridement tip.

15. The apparatus of claim 14, wherein said shield is substantially transparent.

16. The apparatus of claim 14, wherein said shield is formed from polyvinylchloride.

17. The apparatus of claim 14, wherein said shield has a Shore A durometer hardness of about 80-95.

18. The apparatus of claim 17, wherein said shield has a Shore A durometer hardness of about 90.

19. A method of debriding a wound, comprising:
providing a shield including a shield body having a substantially central aperture formed therein, said shield body extending radially from said aperture, said shield further including a rib extending into said aperture;
providing a debridement tip operable to direct an amount of irrigation fluid from a source of irrigation fluid onto an area;
inserting the debridement tip into the central aperture of the shield, whereby the rib forms a press fit with the debridement tip;
positioning the shield between an operator and the wound; and
debriding the wound.

20. The method of claim 19, wherein the debriding step comprises the step of pivoting the debridement tip about an axis transverse to a longitudinal axis of the debridement tip.

21. The method of claim 19, further comprising the step of viewing the wound through the shield during the debriding step.

22. A method of debriding a wound, comprising:
providing a shield including a shield body having a substantially central aperture formed therein, said shield body extending radially from said aperture;
providing a debridement tip operable to direct an amount of irrigation fluid from a source of irrigation fluid onto an area;
inserting the debridement tip into the central aperture of the shield;
positioning the shield between an operator and the wound; and
debriding the wound, while pivoting the debridement tip about an axis transverse to a longitudinal axis of the debridement tip, and concurrently maintaining a substantially static position of said shield body.

23. The method of claim 22, further comprising the step of viewing the wound through the shield during the debriding step.
